(19) United States
(12) Patent Application Publication
(10) Pub. No.: US 2010/0030174 A1
(43) Pub. Date: Feb. 4, 2010

(54) MULTI-PLY FIBROUS STRUCTURES AND PROCESSES FOR MAKING SAME

(76) Inventors: Patrick J. Buschur, Fairfield, OH (US); Kevin Benson McNeil, Loveland, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/185,477

(22) Filed: Aug. 4, 2008

(51) Int. Cl. A61F 13/15; D21H 27/30; D21H 25/04; B05C 1/10

(52) U.S. Cl. 604/365; 604/367; 604/378; 162/132; 162/109; 118/264

(57) ABSTRACT

Multi-ply fibrous structures, more particularly to embossed multi-ply fibrous structures and processes for making same are provided.

### FIELD OF THE INVENTION

The present invention relates to multi-ply fibrous structures, more particularly to embossed multi-ply fibrous structures and processes for making same.

### BACKGROUND OF THE INVENTION

Multi-ply fibrous structures wherein two or more fibrous structure plies are adhesively bonded together (“plybonded”) are known in the art. Examples of known plybonding techniques, especially partial plybonding techniques for embossed multi-ply fibrous structures are described below.

As shown in FIG. 1, in one prior art plybonding technique 10, an embossing patterned roll 12 may be made with some emboss elements at a greater radial distance from the roll center than other emboss elements thus resulting in higher embossments elements 14 and lower embossment elements 16 in an embossed fibrous structure 18. Only the higher embossment elements 14 are contacted with an adhesive 20 via a smooth applicator roll, 22. As the embossed fibrous structure 18 is combined with another fibrous structure 24 and passed through a marrying nip formed by a marrying roll 26 and the embossing patterned roll 12, only the higher embossment elements 14 are bonded. This is because the smooth applicator roll surface 28 carrying the adhesive 20 cannot simultaneously contact the higher embossment elements 14 and the lower embossment elements 16—hence only the embossed fibrous structure areas corresponding to the higher embossment elements 14 have adhesive 20 applied to them. This approach has significant limitations including patterned roll fabrication complexity and variation in embossment clarity (the higher embossment elements 14 are typically exposed to higher pressure than the lower embossment elements 16, such as in a rubber-steel emboss nip).

As shown in FIG. 2, in another prior art plybonding technique 30, a patterned applicator roll 32 may be used to apply adhesive 20 to an embossed fibrous structure 18. The patterned applicator roll 32 comprises protrusions 34 and recesses 36. The protusions 34 may be arranged to correspond to an embossment 38 present in an embossed fibrous structure 18. Adhesive 20 is then only transferred from a gravure roll (not shown) to the patterned applicator roll protrusions 34. The adhesive 20 is then only transferred to an embossed fibrous structure 18 where the embossed fibrous structure 18 contacts the protrusions 34. By registering a desired pattern on the patterned applicator roll 32 with the emboss pattern on an embossed fibrous structure 18, bonding may then be limited to a portion of the embossed area as the embossed fibrous structure 18 is carried on the embossing patterned roll 40. Limitations of this approach are hygiene issues on the patterned applicator roll (glue, dust, and paper build up in the relieved areas) and inadequate plybond if sufficient emboss area is not bonded (the portion of the emboss area that needs to be bonded may be relatively high since the amount of adhesive carried on the surface of the patterned applicator roll is typically relatively low).

As shown in FIG. 3, in another prior plybonding technique 42, an adhesive 20 may be sprayed onto a fibrous structure 44 by a spray nozzle 46. Because the spray application is non-contact and a continuous stream of adhesive 20 is applied to the fibrous structure 44, registration to an emboss pattern in a fibrous structure is not feasible. Limitations with this approach include poor emboss appearance retention (since very little of the emboss area is bonded), flattened emboss appearance if the sheets are laminated in a calendar, and inefficient/costly adhesive utilization in nested and knob-knob configurations (since most of the glue will not correspond to emboss areas and will therefore not be exposed to laminating pressure, and therefore will not result in effective bonding).

As shown in FIG. 4, in even yet another prior art plybonding technique 48, partial plybonding may be achieved in a nested embossing configuration by using a patterned marrying roll 50 that applies a compressive force to two or more fibrous structure plies 52, 54 to achieve bonding between the two or more fibrous structure plies 52, 54 to form a multi-ply fibrous structure 56. In such a configuration, portions of the patterned marrying roll 50 comprise protrusions 58 and other portions are recessed 60. The protrusions 58 of the patterned marrying roll 50 contact a portion of the emboss elements 62 on an embossing patterned roll 64. The portions of the embossing pattern which correspond to the relieved portion of the patterned marrying roll 50 are not subject to laminating pressure and are thus not bonded. Limitations of this approach are hygiene issues on the patterned marrying roll 50 (glue, dust, and paper build up in the relieved areas) and inadequate plybond if sufficient emboss area is not bonded (the portion of the emboss area that needs to be bonded may be relatively high since the amount of adhesive carried on the surface of the applicator roll is typically relatively low), and inefficient/costly adhesive utilization (since most of the glue will not correspond to emboss areas and will therefore not be exposed to laminating pressure, and therefore will not result in effective bonding).

There exists a need for a new and/or improved plybonding technique for fibrous structures that overcomes the negatives associated with prior art plybonding techniques.

### SUMMARY OF THE INVENTION

The present invention fulfills the need described above by providing a plybonding process for making a new multi-ply fibrous structure.

In one example of the present invention, a multi-ply fibrous structure comprising two or more fibrous structure plies plybonded to one another such that the multi-ply fibrous structure exhibits a ratio of plybond strength to plybond area of the multi-ply fibrous structure of greater than 100 g/in/% plybond area, is provided.

In another example of the present invention, a multi-ply sanitary tissue product comprising a multi-ply fibrous structure according to the present invention, is provided.

In even another example of the present invention, a process for making a multi-ply fibrous structure, the process comprising the step of associating a first fibrous structure ply with a second fibrous structure ply such that a multi-ply fibrous structure that exhibits a ratio of plybond strength to plybond area of greater than 100 g/in/% plybond area is formed, is provided.

In still another example of the present invention, a process for making a multi-ply fibrous structure, the process comprising the step of applying a compressive force of less than about 80 pli to plybond a first fibrous structure ply to a second fibrous structure ply, is provided.

In yet another example of the present invention, a process for making a multi-ply fibrous structure, the process comprising the step of compressing a first fibrous structure ply and a second fibrous structure ply together with a compressive force of less than 40 pli, such that a multi-ply fibrous structure exhibits a plybond strength of greater than 0.5 g/in.

In still yet another example of the present invention, a process for making a multi-ply fibrous structure, the process comprising the steps of:

a. plybonding a first fibrous structure ply and a second fibrous structure together with an adhesive exhibiting a viscosity of greater than 500 centipoise and/or greater than 1,000 centipoise and/or greater than 5,000 centipoise and/or greater than 10,000 centipoise to form a multi-ply fibrous structure; and

b. applying a compressive force to the multi-ply fibrous structure that exhibits a plybond strength of greater than 0.5 g/in, is provided.

In still another example of the present invention, an adhesive application system comprising a permeable roll comprising an interior volume comprising an adhesive, wherein the interior volume is in fluid communication via a plurality of holes with an external environment comprising a fibrous structure comprising embossments, wherein the holes are arranged such that the adhesive is applied to less than the entire area of embossments, is provided.

Accordingly, the present invention provides a multi-ply fibrous structure, a process for making such a multi-ply fibrous structure, a multi-ply sanitary tissue product comprising such a multi-ply fibrous structure, and an adhesive application system.

### BRIEF DESCRIPTION OF THE DRAWINGS

FIG. 1 is a schematic representation of a prior art plybonding technique;

FIG. 2 is a schematic representation of a prior art plybonding technique;

FIG. 3 is a schematic representation of a prior art plybonding technique;

FIG. 4 is a schematic representation of a prior art plybonding technique;

FIG. 5 is a perspective view of a multi-ply fibrous structure according to the present invention;

FIG. 6 is a cross-sectional view of the multi-ply fibrous structure of FIG. 5 taken along line 6-6;

FIG. 7 is a partial, cross-sectional view of a process for making a multi-ply fibrous structure;

FIG. 8 is a schematic representation of a process for making multi-ply fibrous structure.

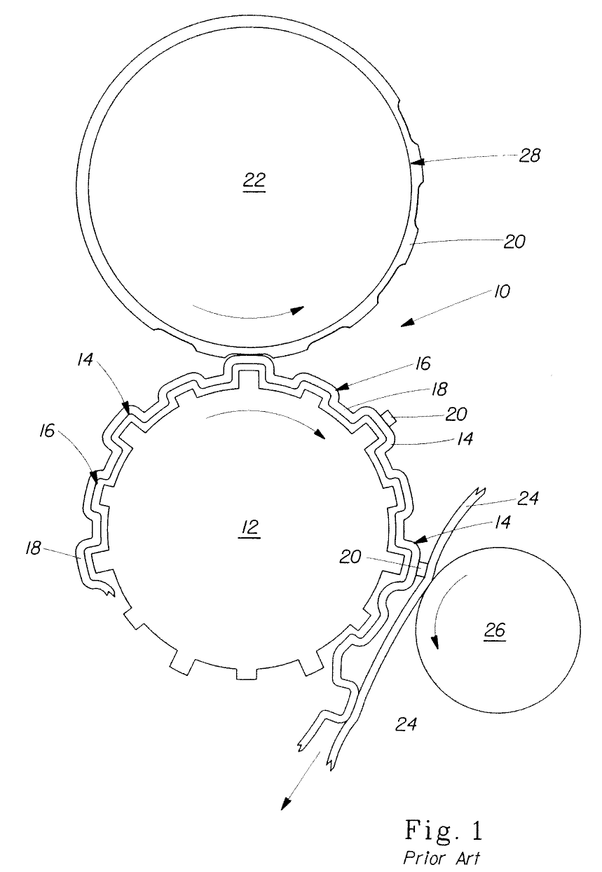

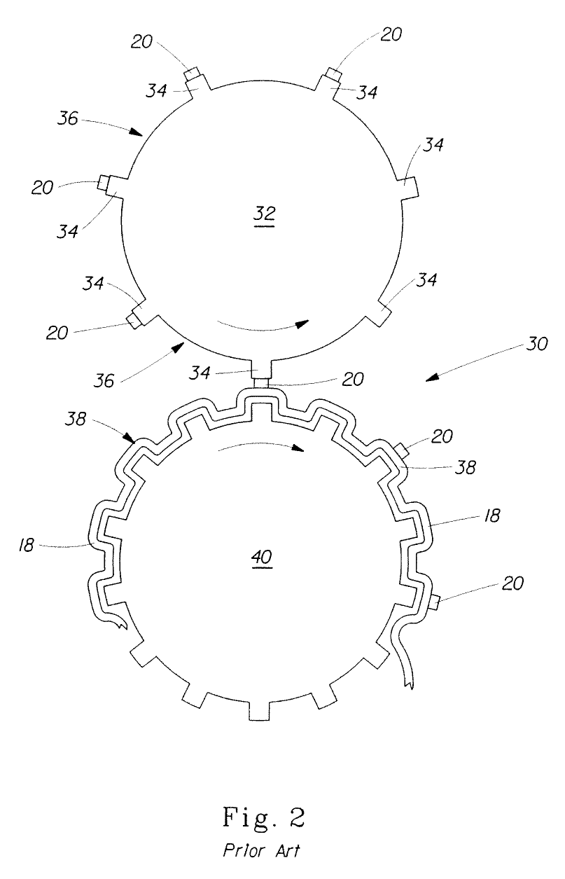

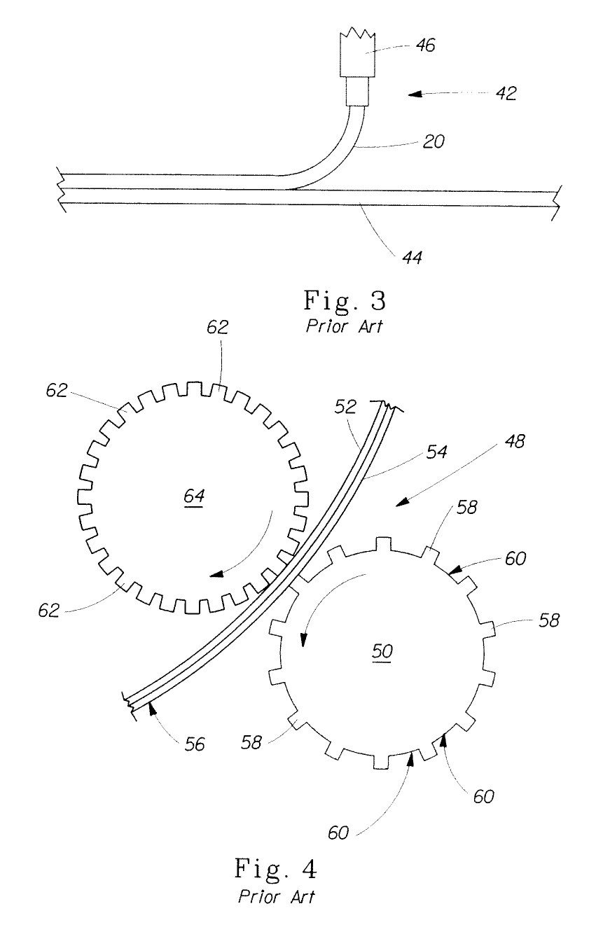

### DETAILED DESCRIPTION OF THE INVENTION

### Definitions

“Fibrous structure” as used herein means a structure that comprises one or more filaments and/or fibers. In one example, a fibrous structure according to the present invention means an orderly arrangement of filaments and/or fibers within a structure in order to perform a function. Nonlimiting examples of fibrous structures of the present invention include paper, fabrics (including woven, knitted, and non-woven), and absorbent pads (for example for diapers or feminine hygiene products).

Nonlimiting examples of processes for making fibrous structures include known wet-laid papermaking processes and air-laid papermaking processes. Such processes typically include steps of preparing a fiber composition in the form of a suspension in a medium, either wet, more specifically aqueous medium, or dry, more specifically gaseous, i.e. with air as medium. The aqueous medium used for wet-laid processes is oftentimes referred to as a fiber slurry. The fibrous slurry is then used to deposit a plurality of fibers onto a forming wire or belt such that an embryonic fibrous structure is formed, after which drying and/or bonding the fibers together results in a fibrous structure. Further processing the fibrous structure may be carried out such that a finished fibrous structure is formed. For example, in typical papermaking processes, the finished fibrous structure is the fibrous structure that is wound on the reel at the end of papermaking, and may subsequently be converted into a finished product, e.g. a sanitary tissue product.

The fibrous structure of the present invention may exhibit a basis weight between about 10 g/m² to about 120 g/m² and/or from about 15 g/m² to about 110 g/m² and/or from about 20 g/m² to about 100 g/m² and/or from about 30 to 90 g/m². In addition, the fibrous structure of the present invention may exhibit a basis weight between about 40 g/m² to about 120 g/m² and/or from about 50 g/m² to about 110 g/m² and/or from about 55 g/m² to about 105 g/m² and/or from about 60 to 100 g/m².

The fibrous structure of the present invention may exhibit a total dry tensile strength of greater than about 59 g/cm (150 g/in) and/or from about 78 g/cm (200 g/in) to about 394 g/cm (1000 g/in) and/or from about 98 g/cm (250 g/in) to about 335 g/cm (850 g/in). In addition, the fibrous structure of the present invention may exhibit a total dry tensile strength of greater than about 196 g/cm (500 g/in) and/or from about 196 g/cm (500 g/in) to about 394 g/cm (1000 g/in) and/or from about 216 g/cm (550 g/in) to about 335 g/cm (850 g/in) and/or from about 236 g/cm (600 g/in) to about 315 g/cm (800 g/in). In one example, the fibrous structure exhibits a total dry tensile strength of less than about 394 g/cm (1000 g/in) and/or less than about 335 g/cm (850 g/in).

In another example, the fibrous structure of the present invention may exhibit a total dry tensile strength of greater than about 196 g/cm (500 g/in) and/or greater than about 236 g/cm (600 g/in) and/or greater than about 276 g/cm (700 g/in) and/or greater than about 315 g/cm (800 g/in) and/or greater than about 354 g/cm (900 g/in) and/or greater than about 394 g/cm (1000 g/in) and/or from about 315 g/cm (800 g/in) to about 1968 g/cm (5000 g/in) and/or from about 354 g/cm (900 g/in) to about 1181 g/cm (3000 g/in) and/or from about 354 g/cm (900 g/in) to about 984 g/cm (2500 g/in) and/or from about 394 g/cm (1000 g/in) to about 787 g/cm (2000 g/in).

The fibrous structure of the present invention may exhibit an initial total wet tensile strength of less than about 78 g/cm (200 g/in) and/or less than about 59 g/cm (150 g/in) and/or less than about 39 g/cm (100 g/in) and/or less than about 29 g/cm (75 g/in).

The fibrous structure of the present invention may exhibit an initial total wet tensile strength of greater than about 118 g/cm (300 g/in) and/or greater than about 157 g/cm (400 g/in) and/or greater than about 196 g/cm (500 g/in) and/or greater than about 236 g/cm (600 g/in) and/or greater than about 276 g/cm (700 g/in) and/or greater than about 315 g/cm (800 g/in) and/or greater than about 354 g/cm (900 g/in) and/or greater than about 394 g/cm (1000 g/in) and/or from about 118 g/cm (300 g/in) to about 1968 g/cm (5000 g/in) and/or from about 157 g/cm (400 g/in) to about 1181 g/cm (3000 g/in) and/or from about 196 g/cm (500 g/in) to about 984 g/cm (2500 g/in) and/or from about 196 g/cm (500 g/in) to about 787 g/cm (2000 g/in) and/or from about 196 g/cm (500 g/in) to about 591 g/cm (1500 g/in).

The fibrous structure of the present invention may exhibit a density (measured at 95 g/in²) of less than about 0.60 g/cm³ and/or less than about 0.30 g/cm³ and/or less than about 0.20 g/cm³ and/or less than about 0.10 g/cm³ and/or less than about 0.07 g/cm³ and/or less than about 0.05 g/cm³ and/or from about 0.01 g/cm³ to about 0.20 g/cm³ and/or from about 0.02 g/cm³ to about 0.10 g/cm³.

The fibrous structure of the present invention may exhibit a total absorptive capacity of according to the Horizontal Full Sheet (HFS) Test Method described herein of greater than about 10 g/g and/or greater than about 12 g/g and/or greater than about 15 g/g and/or from about 15 g/g to about 50 g/g and/or to about 40 g/g and/or to about 30 g/g.

The fibrous structure of the present invention may exhibit a Vertical Full Sheet (VFS) value as determined by the Vertical Full Sheet (VFS) Test Method described herein of greater than about 5 g/g and/or greater than about 7 g/g and/or greater than about 9 g/g and/or from about 9 g/g to about 30 g/g and/or to about 25 g/g and/or to about 20 g/g and/or to about 17 g/g.

The fibrous structure of the present invention may be in the form of fibrous structure rolls. Such fibrous structure rolls may comprise a plurality of connected, but perforated sheets of fibrous structure, that are separably dispensable from adjacent sheets. In one example, one or more ends of the roll of fibrous structure may comprise an adhesive and/or dry strength agent to mitigate the loss of fibers, especially wood pulp fibers from the ends of the roll of fibrous structure.

The fibrous structure of the present invention may comprise one or more additives such as softening agents, temporary wet strength agents, permanent wet strength agents, bulk softening agents, lotions, silicones, wetting agents, latexes, especially surface-pattern-applied latexes, dry strength agents such as carboxymethylcellulose and starch, and other types of additives suitable for inclusion in and/or on fibrous structure.

“Fiber” and/or “Filament” as used herein means an elongate particulate having an apparent length greatly exceeding its apparent width, i.e. a length to diameter ratio of at least about 10. For purposes of the present invention, a “fiber” is an elongate particulate as described above that exhibits a length of less than 5.08 cm (2 in.) and a “filament” is an elongate particulate as described above that exhibits a length of greater than or equal to 5.08 cm (2 in.).

Fibers are typically considered discontinuous in nature. Nonlimiting examples of fibers include wood pulp fibers and synthetic staple fibers such as polyester fibers.

Filaments are typically considered continuous or substantially continuous in nature. Filaments are relatively longer than fibers. Nonlimiting examples of filaments include meltblown and/or spunbond filaments. Nonlimiting examples of materials that can be spun into filaments include natural polymers, such as starch, starch derivatives, cellulose and cellulose derivatives, hemicellulose, hemicellulose derivatives, and synthetic polymers including, but not limited to polyvinyl alcohol filaments and/or polyvinyl alcohol derivative filaments, and thermoplastic polymer filaments, such as polyesters, nylons, polyolefins such as polypropylene filaments, polyethylene filaments, and biodegradable or compostable thermoplastic fibers such as polylactic acid filaments, polyhydroxyalkanoate filaments and polycaprolactone filaments. The filaments may be monocomponent or multicomponent, such as bicomponent filaments.

In one example of the present invention, “fiber” refers to papermaking fibers. Papermaking fibers useful in the present invention include cellulosic fibers commonly known as wood pulp fibers. Applicable wood pulps include chemical pulps, such as Kraft, sulfite, and sulfate pulps, as well as mechanical pulps including, for example, groundwood, thermomechanical pulp and chemically modified thermomechanical pulp. Chemical pulps, however, may be preferred since they impart a superior tactile sense of softness to tissue sheets made therefrom. Pulps derived from both deciduous trees (hereinafter, also referred to as “hardwood”) and coniferous trees (hereinafter, also referred to as “softwood”) may be utilized. The hardwood and softwood fibers can be blended, or alternatively, can be deposited in layers to provide a stratified web. U.S. Pat. No. 4,300,981 and U.S. Pat. No. 3,994,771 are incorporated herein by reference for the purpose of disclosing layering of hardwood and softwood fibers. Also applicable to the present invention are fibers derived from recycled paper, which may contain any or all of the above categories as well as other non-fibrous materials such as fillers and adhesives used to facilitate the original papermaking.

In addition to the various wood pulp fibers, other cellulosic fibers such as cotton linters, rayon, lyocell and bagasse can be used in this invention. Other sources of cellulose in the form of fibers or capable of being spun into fibers include grasses and grain sources.

“Sanitary tissue product” as used herein means a soft, low density (i.e. <about 0.15 g/cm3) web useful as a wiping implement for post-urinary and post-bowel movement cleaning (toilet tissue), for otorhinolaryngological discharges (facial tissue), and multi-functional absorbent and cleaning uses (absorbent towels). The sanitary tissue product may be convolutedly wound upon itself about a core or without a core to form a sanitary tissue product roll.

“Weight average molecular weight” as used herein means the weight average molecular weight as determined using gel permeation chromatography according to the protocol found in Colloids and Surfaces A. Physico Chemical & Engineering Aspects, Vol. 162, 2000, pg. 107-121.

“Basis Weight” as used herein is the weight per unit area of a sample reported in lbs/3000 ft² or g/m².

“Machine Direction” or “MD” as used herein means the direction parallel to the flow of the fibrous structure through the fibrous structure making machine and/or sanitary tissue product manufacturing equipment.

“Cross Machine Direction” or “CD” as used herein means the direction parallel to the width of the fibrous structure making machine and/or sanitary tissue product manufacturing equipment and perpendicular to the machine direction.

“Ply” as used herein means an individual, integral fibrous structure.

“Plies” as used herein means two or more individual, integral fibrous structures disposed in a substantially contiguous, face-to-face relationship with one another, forming a multi-ply fibrous structure and/or multi-ply sanitary tissue product. It is also contemplated that an individual, integral fibrous structure can effectively form a multi-ply fibrous structure, for example, by being folded on itself.

“Plybond Strength” as used herein means tensile strength of the multi-ply fibrous structure as measured by the Plybond Strength Test Method described herein.

“Plybond Area” as used herein means the total area of the multi-ply fibrous structure that is bonded together as measured by the Plybond Area Test Method described herein.

As used herein, the articles “a” and “an” when used herein, for example, “an anionic surfactant” or “a fiber” is understood to mean one or more of the material that is claimed or described.

All percentages and ratios are calculated by weight unless otherwise indicated. All percentages and ratios are calculated based on the total composition unless otherwise indicated.

Unless otherwise noted, all component or composition levels are in reference to the active level of that component or composition, and are exclusive of impurities, for example, residual solvents or by-products, which may be present in commercially available sources.

### Multi-Ply Fibrous Structure

The multi-ply fibrous structure of the present invention comprises two or more fibrous structure plies bonded, for example in a face-to-face relationship, to one another such that the multi-ply fibrous structure exhibits a ratio of plybond strength, as measured according to the Plybond Strength Test Method described herein, to plybond area, as measured according to the Plybond Area Test Method described herein, of greater than 100 g/in/% plybond area and/or greater than about 150 g/in/% plybond area and/or greater than about 175 g/in/% plybond area and/or greater than about 200 g/in/% plybond area and/or greater than about 225 g/in/% plybond area. In one example, the multi-ply fibrous structure of the present invention exhibits a ratio of plybond strength to plybond area of greater than 100 g/in/% plybond area to about 1000 g/in/% plybond area and/or from about 150 g/in/% plybond area to about 750 g/in/% plybond area and/or from about 175 g/in/% plybond area to about 500 g/in/plybond area and/or from about 175 g/in/% plybond area to about 400 g/in/% plybond area.

The multi-ply fibrous structure of the present invention may exhibit any suitable plybond strength and plybond area so long as the ratio of plybond strength to plybond area is greater than 100 g/in/% plybond area and/or greater than about 150 g/in/% plybond area and/or greater than about 175 g/in/% plybond area and/or greater than about 200 g/in/% plybond area and/or greater than about 225 g/in/% plybond area.

In one example, the multi-ply fibrous structure exhibits a plybond strength of greater than about 0.5 g/in and/or greater than about 0.75 g/in and/or greater than about 1.0 g/in and/or greater than about 1.5 g/in and/or greater than about 2.0 g/in. In another example, the multi-ply fibrous structure of the present invention exhibits a plybond area of less than about 10% and/or less than about 7% and/or less than about 5% and/or less than about 3% and/or less than about 1% and/or less than about 0.75% and/or greater than about 0.05% and/or greater than about 0.1% and/or greater than about 0.3% and/or greater than about 0.5%. In one example, the multi-ply fibrous structure comprises embossments, wherein all or substantially all of the embossments are plybonded with an adhesive. In another example, less than 60% of the total area of embossments is plybonded with an adhesive.

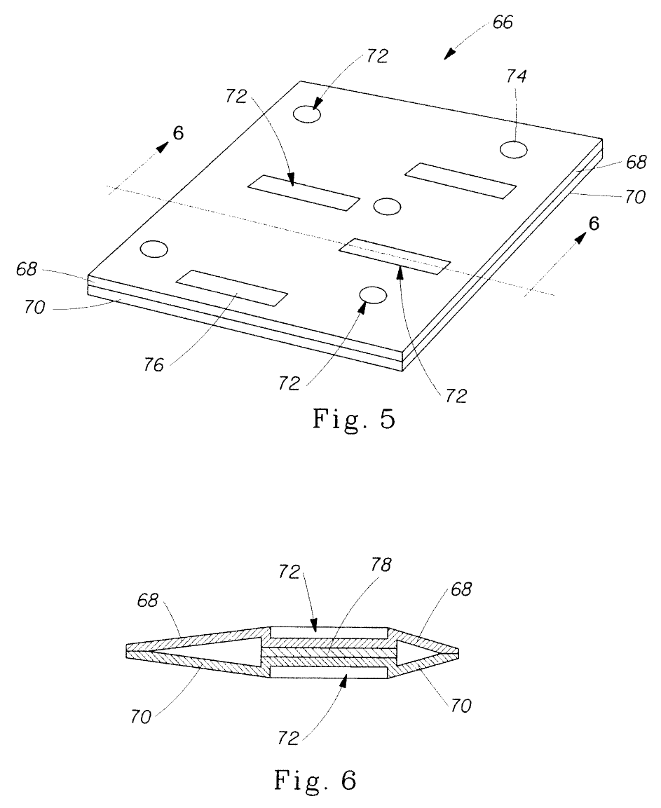

In one example, as shown in FIGS. 5 and 6, a multi-ply fibrous structure 66 of the present invention comprises a first fibrous structure ply 68 and a second fibrous structure ply 70. The first fibrous structure ply 68 and second fibrous structure ply 70 are bonded together with a resulting plybond strength of 1.5 g/in as measured by the Plybond Strength Test Method described herein. The plybond area of the multi-ply fibrous structure 66 is 0.62% as measured by the Plybond Area Test Method described herein. The multi-ply fibrous structure 66 therefore has 242 g/in/% plybond area (calculated as 1.5/0.0062=242) and forms the multi-ply fibrous structure 66 that exhibits a ratio of plybond strength to plybond area of the multi-ply fibrous structure of greater than 100 g/in/% plybond area. The multi-ply fibrous structure 66 may comprise one or more embossments 72. At least one of the embossments 72 may be a dot embossment 74 and/or a line element embossment 76. In one example, the multi-ply fibrous structure 66 comprises at least one dot embossment 74 and at least one line element embossment 76. In one example, an adhesive 78 bonds the first fibrous structure ply 68 and second fibrous structure ply 70 together at at least one embossment 72. The adhesive 78 may be present on one or more embossments 72. In another example, the multi-ply fibrous structure 66 may comprise a plurality of embossments 72, wherein an adhesive 78 may be present on less than all of the embossments 72. The embossments in one or more of the fibrous structure plies of a multi-ply fibrous structure of the present invention may be made by any suitable process. The emboss pattern of the first and second fibrous structure plies may be complementary.

The multi-ply fibrous structure of the present invention may comprise one or more embossments having an embossment height of greater than 200 μm and/or greater than about 300 μm and/or greater than about 400 μm.

In one example, an embossed fibrous structure wherein less than about 60% and/or less than 50% and/or less than 40% and/or greater than 5% and/or greater than 10% of the total embossment area in the embossed fibrous structure is plybonded.

In another example, an embossed fibrous structure of the present invention comprises embossments wherein all of the embossments are plybonded by an adhesive. In yet another example, an embossed fibrous structure of the present invention comprises embossments wherein only a portion of the total embossments are plybonded by an adhesive. In even another example, an embossed fibrous structure of the present invention comprises embossments wherein only portions (not in entirety) of each embossment are plybonded by an adhesive. In still another example, an embossed fibrous structure of the present invention comprises embossments wherein only portions (not in entirety) of a portion of the total embossments are plybonded by an adhesive. In one example, an embossed fibrous structure comprises line element embossments wherein only portions of each line element embossment are plybonded by an adhesive. In yet another example, an embossed fibrous structure comprises line element embossments wherein only portions of a portion of the total line element embossments are plybonded by an adhesive. In still yet another example, an embossed fibrous structure comprises dot embossments wherein only portions of each dot embossment are plybonded by an adhesive. In even yet another example, an embossed fibrous structure comprises dot embossments wherein only portions of a portion of the total dot embossments are plybonded by an adhesive.

### Process for Making a Multi-Ply Fibrous Structure

The multi-ply fibrous structure of the present invention may be made by any suitable process known in the art so long as the multi-ply fibrous structure exhibits a ratio of plybond strength to plybond area of greater than 100 g/in/% plybond area.

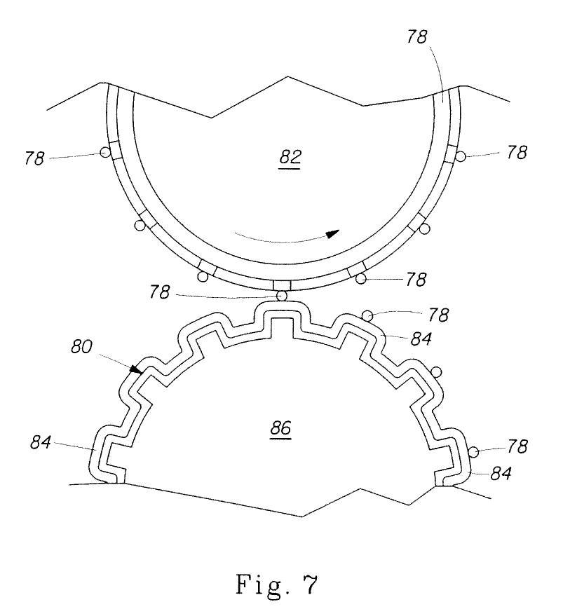

In one example, as shown in FIG. 7, a multi-ply fibrous structure according to the present invention may be made by a process comprising the step of delivering an adhesive 78 to a surface of a fibrous structure, for example an embossed fibrous structure 80, using a permeable roll applicator 82 that contains adhesive 78. The permeable roll applicator 82 may be designed to deposit adhesive 78 on embossments 84, or a portion of the embossments 84 or portions of a portion of the embossments 84 of the embossed fibrous structure 80, for example as the embossed fibrous structure 80 is in contact with a patterned embossing roll 86. Once the fibrous structure, for example the embossed fibrous structure 80, contains the adhesive 78, the fibrous structure is bonded via the adhesive 78 to another fibrous structure (not shown) and passes through a marrying roll nip (not shown) which applies a compressive force to the bonded fibrous structure (not shown).

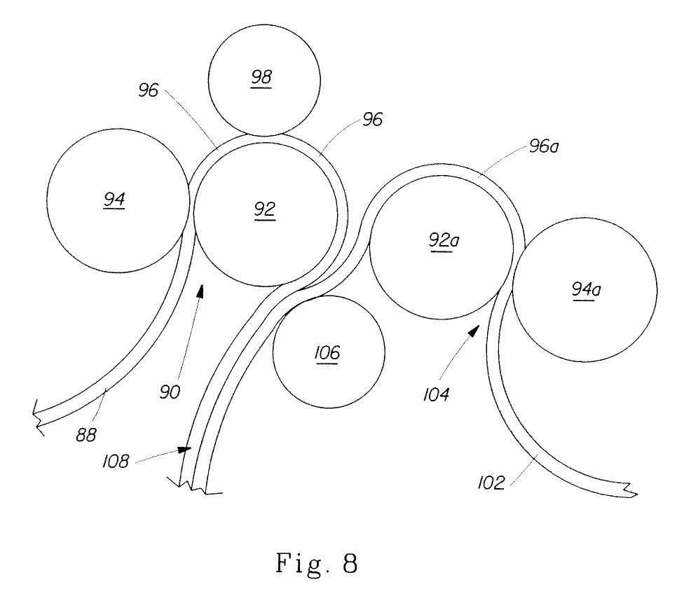

In one example of a process for making a multi-ply fibrous structure, as represented in FIG. 8, a first fibrous structure 88 is embossed by embossing nip 90 formed by a first patterned embossing roll 92 and a second patterned embossing roll 94 to produce an embossed fibrous structure 96. An adhesive (not shown) is then applied to portions of a portion of the individual embossments present in the embossed fibrous structure 96 by an applicator roll 98, which may be permeable fluid applicator roll. A second fibrous structure 102 is embossed by embossing nip 104 formed by a first patterned embossing roll 92′ and a second patterned embossing roll 94′ to produce an embossed fibrous structure 96′. The embossed fibrous structure 96 and embossed fibrous structure 96′ are brought into proximity of one another and are compressed together upon loading of a marrying roll 106 against the first patterned embossing roll 92 via pneumatic or hydraulic or other suitable means to achieve the desired compressive force. This compressive force bonds the two embossed fibrous structures 96, 96′ (plies) together while the embossed fibrous structure 96 is still positioned in its embossing location—that is, the embossments of the embossed fibrous structure 96 are still located on the corresponding emboss pattern elements (for example, protrusions) on the first patterned embossing roll 92 and the adhesive that has been applied to the embossed fibrous structure 96 is also still aligned with the emboss pattern elements (for example, protrusions). The compressive force thus bonds the two embossed fibrous structures 96, 96′ together to form an embossed multi-ply fibrous structure 108.

In one example, the holes of the permeable roll may be registered with other features in a fibrous structure ply making up the multi-ply fibrous structure of the present invention prior to the plybonding process. The holes in the permeable roll may all be the same shape and/or diameter or two or more of the holes may be different shapes and/or different diameters. Further, the length and diameter ratio for a particular hole in the permeable roll may be selected to provide desired adhesive flow characteristics.

Adhesive may be applied as discrete drops, as an essentially continuous pattern of adhesive or any combination thereof onto a fibrous structure, such as an embossed fibrous structure. Typically, at least one fibrous structure is embossed and then at least one fibrous structure is passed by an adhesive application zone where adhesive is applied to a portion of the fibrous structure. Typically, the embossed fibrous structure remains on the embossing pattern roll as the roll rotates past the embossing nip. The fibrous structure is typically deformed in the z-direction such that after the embossing nip, portions of the fibrous structure between embossing elements are deformed down into the relieved portion of the embossing pattern roll, leaving the embossments on the fibrous structure as the most outward oriented portion of the fibrous structure on the periphery of the embossing pattern roll.

In one example, adhesive is applied to a fibrous structure, such as an embossed fibrous structure using a permeable roll. Adhesive is delivered from the interior of the permeable roll as it rotates and as the fibrous structure comes into contact with the adhesive from the permeable roll.

By utilizing the permeable roll to apply adhesive to a fibrous structure, a wider range of adhesive viscosities can be used compared to known adhesive application systems. Adhesives that exhibit higher viscosities and/or higher solids content provide high adhesion and can thus enable achieving adequate plybond with very low adhesive add-on rates and relatively low lamination pressures. In addition to the adhesion properties, the permeable roll system also provides better hygiene conditions since it minimizes dust transfer to any interfacing rolls, for example a patterned roll, and/or adhesive applicator system during operation.

The adhesive may be applied intermittently to the fibrous structure. Further, the rate of application of the adhesive may be controlled by fibrous structure speed, fibrous structure material characteristics and/or properties, fibrous structure tension and combinations thereof.

In one example of the present invention, a low pressure marrying roll nip (for example, less than about 40 pli and/or less than about 20 pli and/or less than about 10 pli to about 1 pli and/or to about 2 pli and/or to about 5 pli) for laminating the fibrous structures together is achievable due to the fact that a higher tack, high viscosity adhesive, such as polyvinyl alcohol (14% solids, 10,000 centipoise) can be used. A non-limiting example of a suitable adhesive is commercially available from Henkel as Henkel 52-6014. Such an adhesive is more effective at creating bond strength than lower tack, low viscosity adhesives, which are typically required to be used in adhesive application processes other than the permeable fluid applicator process. As a result, the amount of compressive force required in the marrying roll nip is significantly less than is required for existing marrying roll nip processes.

In one example, all embossing, adhesive applying and marrying roll nips according to the present invention exhibit a pressure of less than about 80 pli and/or less than about 60 pli and/or less than about 40 pli and/or less than about 25 pli and/or less than about 15 pli and/or less than about 10 pli and/or greater than 1 pli and/or greater than 5 pli.

After laminating (bonding two or more fibrous structure plies together), the multi-ply fibrous structure can be conveyed to other fibrous structure processing stations such as lotioning, coating, printing, slitting, folding, perforating, winding, tuft-generating, and the like. Alternatively, some of these other fibrous structure processing transformations may occur prior to the laminating (bonding) transformations.

### Non-Limiting Example of a Multi-Ply Fibrous Structure

A multi-ply fibrous structure according to the present invention is made by bonding two fibrous structure plies, for example two embossed through-air-dried fibrous structure plies, together such that the multi-ply fibrous structure exhibits a ratio of plybond strength to plybond area of about 242 g/in/% plybond area and a plybond strength of about 1.5 g/in and/or a plybond area of about 0.62%, which in this case happens to be about 10% of the total embossed area of the multi-ply fibrous structure.

### Permeable Roll Applicator

A permeable roll of the present invention comprises an interior volume that is in fluid communication with an exterior environment. In one example, the permeable roll comprises shell having an interior surface and an exterior surface connected to one another via one or more holes which permits fluid communication from the interior volume to an external environment.

The permeable roll of the present invention may have an outer diameter that is the same as the outer diameter of any patterned embossing rolls (30.48 cm (12.00 inches) in this example) associated with the permeable roll. The permeable roll may have a stainless steel shell thickness of 0.3175 cm (0.125 inch). The discrete holes in the permeable roll shell through which adhesive flows are approximately 0.2032 mm (0.008 inch) in diameter.

A typical shell fabrication process includes making a 0.3175 cm (0.125 inch) thick shell from carbon composite material and then mechanically drilling holes with the target 0.2032 mm (0.008 inch) diameter. Another shell fabrication process includes making a 0.3175 cm (0.125 inch) thick shell from carbon composite material and then mechanically drilling holes with the target 0.1524 cm (0.060 inch) diameter, then spin casting epoxy or resin to file the holes, allowing the epoxy and/or resin to cure, and then mechanically redrilling holes with the target 0.2032 mm (0.008 inch) diameter.

An alternative shell fabrication process includes making a sand casting of a shell with greater thickness than required in final form, grinding the shell to a thickness of 0.6858 cm (0.27 inch), trepan drilling via laser, holes which extend from the shell inside surface to the shell outside surface in desired locations with a diameter of 0.1016 cm (0.040 inch), plasma spray coating a stainless steel based coating on the outer surface of the shell at a thickness greater than 0.1016 cm (0.040 inch), grinding the outer surface of the shell to achieve a total thickness of 0.7874 cm (0.31 inch), and then laser drilling holes with a diameter of 0.2032 mm (0.008 inch) that extend from the outer surface of the shell's plasma spray coating to the inner surface of the plasma spray coating such that the 0.2032 mm (0.008 inch) diameter hole is aligned with and completely within the 0.1016 cm (0.040 inch) diameter hole.

Other fabrication techniques as known to one of skill in the art may be used, including drilling via laser or electron beam, using concentric shells wherein the outer shell is thinner (allowing easier drilling of relatively small diameter holes) than the inner shell and is heat shrunk on the inner shell (which has been pre-drilled with larger holes which will be aligned with new holes in the thin outer shell), or other suitable techniques. Non-limiting processes for creating holes in the roll to make it a permeable roll include laser drilling, electron beam drilling, mechanical drilling, electrical discharge machining drilling, chemical engraving, metalizing processes, sintering processes, castings and combinations thereof.

Adhesive flow through the holes in the permeable roll during operation (i.e., during rotating of the permeable roll) may be directly controlled via a positive displacement pump and/or pressure regulation at the inlet to the hole within the interior surface of the permeable roll which is balanced with the pressure drop through the hole and results in a controlled flow rate. The size and pattern of the holes may be designed to provide a desired add-on rate and adhesive application pattern for a chosen adhesive and viscosity with minimum air entrainment into the adhesive application system. Adhesive flow can be adjusted on the run via adjustment of the positive displacement pump speed and/or internal pressure and may be controlled in relation to other system parameters such as line speed, operating temperature, and the like.

The holes in the permeable roll may be fabricated in any desired pattern and the permeable roll circumference and drive can be designed to match/register other process transformations, such as embossing. For example, adhesive may be applied in phased relationship to other fluids, perforations, cross machine fibrous structure edges, embossing, printing, etc. Phasing may be achieved via close coupling (direct gear drive for adjacent rolls performing different transformations), web handling/feed rate matching, closed loop control with sensing of the adhesive and other transformations in the fibrous structure, or any other suitable means. This phasing potential enables more efficient utilization of adhesives in the fibrous structure product design, thereby improving quality while minimizing adhesive cost, and enables potential synergies between transformations (e.g. highlight emboss or perforations, complement printing, etc.).

The holes in a permeable roll may be designed to match portions or all of embossments in an embossed fibrous structure. The permeable roll and the embossing roll, which imparts the embossments to the fibrous structure, may have the same diameter and circumference or a multiple of each other which may result in a mismatched speed at transfer but would still stay in phase, or it is possible to have rolls of different diameter but the emboss pattern, adhesive pattern and/or roll diameters may be such that they all stay in phase either with matched speed or mismatched speed. The permeable roll can be positioned adjacent to the embossing roll and driven in synchronization with the embossing roll via direct gear drive, timing belt, linked servo drives, and/or other suitable means to apply drops of adhesive on a desired portion of embossments in a fibrous structure after embossing and prior to the embossed fibrous structure being joined with another fibrous structure and compressed/laminated together in a marrying nip. In this manner a desired 2-ply embossed and laminated product can be produced wherein the adhesive laminating fluid is phased to embossing. This can create an embossed product having a greater softness because adhesive is only applied to the embossed fibrous structure where needed, the adhesive is also only applied to recessed embossed areas of the fibrous structure (once in sanitary tissue product form), and the adhesive zones in the fibrous structure have a relatively high volume of adhesive, thereby enabling sufficient plybond strength even with a relatively low plybonded area (less plybonded area enhances fibrous structure flexibility, a key element in consumer softness perception).

The holes in the permeable roll may be registered to an emboss pattern present on an embossed fibrous structure to provide adhesive application only on embossments or a portion of the embossments within the embossed fibrous structure. For example, adhesive may be applied to only to a portion of dot and/or line element embossments present in an embossed fibrous structure as a result of the holes of the permeable roll that applies the adhesive being registered to only a portion of the dot and/or line element embossments such that only portions of the embossments and/or portions of portions of the embossments are plybonded. Further, the adhesive may be delivered from the permeable roll at any rate. For example, an adhesive may be forced through the holes of the permeable roll by a positive displacement pump, such as a Seepex positive displacement pump, and/or pressure regulation at a rate of 0.013 g/m² in the fibrous structure or about 0.003 g/minute/hole when operating a fibrous structure application speed of about 2000 feet per minute.

The permeable roll may be made by any suitable materials. Non-limiting examples of suitable materials for the permeable roll include steel, aluminum, other metals, carbon composite materials, plastics, natural rubber, synthetic rubber and/or other materials that provide a relatively rigid surface comprising holes for application flow.

The permeable roll may be fabricated as a single unit or may comprise sleeve sections (both in the circumferential and cross machine directions) that combine to form the permeable roll.

### Test Method

Unless otherwise indicated, all tests described herein including those described under the Definitions section and the following test methods are conducted on samples, test equipment and test surfaces that have been conditioned in a conditioned room at a temperature of 73° F.±4° F. (about 23° C.±2.2° C.) and a relative humidity of 50%±10% for 12 hours prior to the test. Further, all tests are conducted in such conditioned room.

### Plybond Strength Test Method

The plybond strength (dry plybond strength for purposes of the present invention) of a multi-ply fibrous structure is measured using a tensile tester. Strips of 7.62 cm wide (3 inch wide) of four samples of a multi-ply fibrous structure running the entire length, but no longer than about 28 cm (about 11 inches) of the samples is cut from the center of each sample. Two of the strips are cut in the machine direction and the other two are cut in the cross machine direction (i.e., between perforations in the machine direction or between edges in the cross machine direction). If the fibrous structure is in roll form, cut the samples from greater than 40″ (1016 mm) from the ends of the roll. The plies of each strip are initially separated from one another about 50 mm (2 inches) along either of the 7.62 cm wide edges, so that each ply is available independent of the other and each ply has a gauge length of 5.08 cm (2 inches). Each ply of a strip is placed in a jaw (grip) of a tensile tester. A suitable tensile tester is an EJA Vantage tensile tester available from Thwing-Albert Instrument Company, West Berlin, N.J. The sample strip needs to be centered in the grips and straight. The crosshead separation speed is set at 20 inches per minute, the test distance is set at 6.5 inches, the load cell is 5000 g and the load divider is set at 3. Activate the tensile tester. When the test is complete, record the load mean value in g/in. Remove the sample strip from the grips and discard. Check the load cell for a zero reading. Repeat the process for each sample strip. Four samples are tested in tension. The four plybond strength numbers are then averaged to give an average plybond strength value in g/in units.

Care must be taken that the portion of the sample yet to be separated by the tensile machine does not contact the lower jaw or the lower crosshead of the tensile machine. If such contact occurs, it will register on the load cell and give a reading which is erroneously high. Similarly, care must be taken that the portion of the sample yet to be separated does not contact the portion of the sample having the plies already separated by the tensile tester. If such contact occurs, it will falsely increase the apparent ply bond strength. If either of the aforementioned contacts occur, the data point is to be discarded and a new sample tested.

Do not use samples that contain obvious defects, such as wrinkles, creases, tears, holes, etc.

### Plybond Area Test Method

The plybond area test used to determine the % plybond area in a multi-ply fibrous structure is set forth below.

A 10.16 cm×10.16 cm (4 in.×4 in.) sample of a multi-ply fibrous structure is prepared. The sample is place on a flat surface. Using a Preval Sprayer (net wt. 2.0 oz) sprayer, available from Precision Valve Corporation, Yonkers, N.Y., an indicator solution is sprayed evenly onto the sample until the indicator solution just wets and penetrates the entire sample (each ply of the sample). If the multi-ply fibrous structure is bonded with an adhesive, a color, such as the color blue, will become noticeable. The indicator solution is made as follows:

1. 0.635 g Iodine crystals and 2.00 g Potassium Iodide are mixed in a 50 ml volumetric flask and diluted with distilled water to the 50 ml mark on the flask. The solution is mixed until the Iodine crystals and Potassium Iodide have dissolved in the distilled water forming a 0.1N Iodine solution. Set the solution aside.

2. 20.00 g Boric Acid is added to a 500 ml volumetric flask and diluted with distilled water to the 500 ml mark on the flask. The solution is mixed until the Boric Acid has dissolved in the distilled water making a 4% Boric Acid solution. Set the solution aside.

3. Transfer 50 ml of 0.1N Iodine solution from 1 above into a 1000 ml volumetric flask. Rinse the Iodine solution flask with distilled water and add to the 1000 ml volumetric flask for full transfer. Add 400 ml of the 4% Boric Acid solution to the 1000 ml volumetric flask by a graduated cylinder. Rinse the graduated cylinder into the 1000 ml volumetric flask with distilled water. Finally, dilute the contents of the 1000 ml volumetric flask to the 1000 ml mark on the flask with distilled water and mix to form the indicator solution to be sprayed on the sample.

Place the wetted sample entirely between two (2) 0.5 cm thick clear (not frosted or textured) glass plates to provide flatness to the sample.

Place a Kaiser RS3 Light stand available from B&H Photo, New York, N.Y., with two Sylvania NU B1 Super Flood BCA bulbs so that the sample is illuminated from the top and side of the sample. Minimize the other lighting to provide the most contrast resulting from the bulbs on the light stand. A Spot Insight Color Camera available from Diagnostic Instruments, Inc., Sterling Heights, Mich., with a Funion-TV 1:1.4/12/5 camera lens available from Fuji Photo Optical Company, Japan, is positioned to take a photo perpendicular to the top of the sample.

The photo of the top of the sample is then analyzed by imaging software either Optimas v 6.51 or ImagePro, both available from Media Cybernetics, Inc., Bethesda, Md.

The color band for analysis via the imaging software is selected for the best contrast for the sample. Images are preprocessed to correct for small variations in lighting by using a local smoothing background correction filter of 30×30 pixels prior to thresholding. The corrected single color band image is thresholded to select only areas of glue. The % plybond area of the multi-ply fibrous structure is obtained from the percent area function of the imaging software.

### Embossment Height Test Method

Embossment height is measured using a GFM Primos Optical Profiler instrument commercially available from GFMesstechnik GmbH, Warthestraβe 21, D14513 Teltow/Berlin, Germany. The GFM Primos Optical Profiler instrument includes a compact optical measuring sensor based on the digital micro mirror projection, consisting of the following main components: a) DMD projector with 1024×768 direct digital controlled micro mirrors, b) CCD camera with high resolution (1300×1000 pixels), c) projection optics adapted to a measuring area of at least 27×22 mm, and d) recording optics adapted to a measuring area of at least 27×22 mm; a table tripod based on a small hard stone plate; a cold light source; a measuring, control, and evaluation computer; measuring, control, and evaluation software ODSCAD 4.0, English version; and adjusting probes for lateral (x-y) and vertical (z) calibration.

The GFM Primos Optical Profiler system measures the surface height of a sample using the digital micro-mirror pattern projection technique. The result of the analysis is a map of surface height (z) vs. xy displacement. The system has a field of view of 27×22 mm with a resolution of 21 microns. The height resolution should be set to between 0.10 and 1.00 micron. The height range is 64,000 times the resolution.

To measure a fibrous structure sample do the following:
1. Turn on the cold light source. The settings on the cold light source should be 4 and C, which should give a reading of 3000K on the display;
2. Turn on the computer, monitor and printer and open the ODSCAD 4.0 Primos Software.
3. Select “Start Measurement” icon from the Primos taskbar and then click the “Live Pic” button.
4. Place a 30 mm by 30 mm sample of fibrous structure product conditioned at a temperature of 73° F.±2° F. (about 23° C.±1° C.) and a relative humidity of 50%±2% under the projection head and adjust the distance for best focus.
5. Click the “Pattern” button repeatedly to project one of several focusing patterns to aid in achieving the best focus (the software cross hair should align with the projected cross hair when optimal focus is achieved). Position the projection head to be normal to the sample surface.
6. Adjust image brightness by changing the aperture on the lens through the hole in the side of the projector head and/or altering the camera “gain” setting on the screen. Do not set the gain higher than 7 to control the amount of electronic noise. When the illumination is optimum, the red circle at bottom of the screen labeled “I.O.” will turn green.
7. Select Technical Surface/Rough measurement type.
8. Click on the “Measure” button. This will freeze on the live image on the screen and, simultaneously, the image will be captured and digitized. It is important to keep the sample still during this time to avoid blurring of the captured image. The image will be captured in approximately 20 seconds.
9. If the image is satisfactory, save the image to a computer file with “.omc” extension. This will also save the camera image file “.kam”.
10. To move the date into the analysis portion of the software, click on the clipboard/man icon.
11. Now, click on the icon “Draw Cutting Lines”. Make sure active line is set to line 1. Move the cross hairs to the lowest point on the left side of the computer screen image and click the mouse. Then move the cross hairs to the lowest point on the right side of the computer screen image on the current line and click the mouse. Now click on “Align” by marked points icon. Now click the mouse on the lowest point on this line, and then click the mouse on the highest point on this line. Click the “Vertical” distance icon. Record the distance measurement. Now increase the active line to the next line, and repeat the previous steps, do this until all lines have been measured (six (6) lines in total. Take the average of all recorded numbers, and if the units is not micrometers, convert it to micrometers (μm). This number is the embossment height. Repeat this procedure for another image in the fibrous structure product sample and take the average of the embossment heights.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

Every document cited herein, including any cross referenced or related patent or application, is hereby incorporated herein by reference in its entirety unless expressly excluded or otherwise limited. The citation of any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A multi-ply fibrous structure comprising two or more fibrous structure plies bonded to one another such that the multi-ply fibrous structure exhibits a ratio of plybond strength to plybond area of the multi-ply fibrous structure of greater than 100 g/in/% plybond area.

2. The multi-ply fibrous structure according to claim 1 wherein the two or more fibrous structure plies are bonded to one another by an adhesive that bonds the two or more fibrous structure plies together to form the multi-ply fibrous structure.

3. The multi-ply fibrous structure according to claim 2 wherein the multi-ply fibrous structure comprises one or more embossments.

4. The multi-ply fibrous structure according to claim 3 wherein at least one of the embossments comprises a dot embossment.

5. The multi-ply fibrous structure according to claim 3 wherein at least one of the embossments comprises a line element embossment.

6. The multi-ply fibrous structure according to claim 3 wherein the multi-ply fibrous structure comprises at least one dot embossment and at least one line element embossment.

7. The multi-ply fibrous structure according to claim 3 wherein an adhesive is present on less than the entire surface area of at least one of the embossments.

8. The multi-ply fibrous structure according to claim 3 wherein the multi-ply fibrous structure comprises a plurality of embossments, wherein the adhesive is present on less than all of the embossments.

9. The multi-ply fibrous structure according to claim 1 wherein the multi-ply fibrous structure exhibits a plybond strength of greater than 0.5 g/in.

10. The multi-ply fibrous structure according to claim 1 wherein the multi-ply fibrous structure exhibits a plybond area of less than 10%.

11. A multi-ply sanitary tissue product comprising a multi-ply fibrous structure according to claim 1.

12. A process for making a multi-ply fibrous structure, the process comprising the step of associating a first fibrous structure ply to a second fibrous structure ply together such that a multi-ply fibrous structure that exhibits a ratio of plybond strength to plybond area of greater than 100 g/in/% plybond area is formed.

13. The process according to claim 12 wherein the two or more fibrous structure plies are bonded to one another by an adhesive that bonds the two or more fibrous structure plies together to form the multi-ply fibrous structure.

14. The process according to claim 13 wherein the multi-ply fibrous structure comprises one or more embossments.

15. The process according to claim 14 wherein at least one of the embossments comprises a dot embossment.

16. The process according to claim 14 wherein at least one of the embossments comprises a line element embossment.

17. The process according to claim 14 wherein the multi-ply fibrous structure comprises at least one dot embossment and at least one line element embossment.

18. The process according to claim 14 wherein an adhesive is present on less than the entire surface area of at least one of the embossments.

19. The process according to claim 14 wherein the multi-ply fibrous structure comprises a plurality of embossments, wherein the adhesive is present on less than all of the embossments.

20. The process according to claim 12 wherein the multi-ply fibrous structure exhibits a plybond strength of greater than 0.5 g/in.

21. The process according to claim 12 wherein the multi-ply fibrous structure exhibits a plybond area of less than 10%.

22. A process for making a multi-ply fibrous structure, the process comprising the step of applying a compressive force of less than about 80 pli to plybond a first fibrous structure ply to a second fibrous structure ply forming a multi-ply fibrous structure.

23. The process according to claim 22 wherein the multi-ply fibrous structure exhibits a plybond strength of greater than 0.5 g/in.

24. A process for making a multi-ply fibrous structure, the process comprising the steps of:
a. plybonding a first fibrous structure ply and a second fibrous structure together with an adhesive exhibiting a viscosity of greater than 500 centipoise to form a multi-ply fibrous structure; and
b. applying a compressive force to the multi-ply fibrous structure that exhibits a plybond strength of greater than 0.5 g/in.

25. An adhesive application system comprising a permeable roll comprising an interior volume comprising an adhesive, wherein the interior volume is in fluid communication via a plurality of holes with an external environment comprising a fibrous structure comprising embossments, wherein the holes are arranged such that the adhesive is applied to less than the entire area of embossments.
